(19) United States
(12) Patent Application Publication
(10) Pub. No.: US 2021/0296586 A1
(43) Pub. Date: Sep. 23, 2021

(54) THERMALLY ACTIVATED DELAYED FLOURESCENCE (TADF) MATERIAL, SYNTHESIZING METHOD THEREOF, AND ELECTROLUMINESCENT DEVICE

(71) Applicant: WUHAN CHINA STAR OPTOELECTRONICS SEMICONDUCTOR DISPLAY TECHNOLOGY CO., LTD., Wuhan, Hubei (CN)

(72) Inventor: Yanjie WANG, Wuhan, Hubei (CN)

(21) Appl. No.: 16/768,676

(22) PCT Filed: Apr. 7, 2020
(86) PCT No.: PCT/CN2020/083472

§ 371 Date: May 31, 2020

(30) Foreign Application Priority Data
Mar. 23, 2020 (CN) 202010209956.8

(51) Int. Cl. H01L 51/00; C09K 11/06; C07C 225/22

(52) U.S. Cl. CPC H01L 51/006; C09K 11/06; C07C 225/22; C09K 2211/1011; H01L 51/5016; H01L 51/0056; C07C 2603/54; H01L 51/0003; H01L 51/0025; C09K 2211/1007

(57) ABSTRACT

A thermally activated delayed fluorescence (TADF) material, a synthesizing method thereof, and an electroluminescent device is provided. The TADF is a target compound synthesized from an electron donator and an electron acceptor. The target compound has a Dₙ-A molecular structure, wherein n denotes 1, 2, or 3, D is the electron donator, and A is the electron acceptor.

### FIELD

The present disclosure relates to the field of display, and more particularly, relates to a thermally activated delayed fluorescence (TADF) material, a synthesizing method thereof, and an electroluminescent device.

### BACKGROUND

Organic light-emitting diodes (OLEDs) have promising prospect in display and luminescent fields, and have attracted attention from scientific researchers and companies due to advantages such as self-luminescence, wide viewing angles, fast response times, wide temperature adaptation ranges, low driving voltage, low power consumption, high brightness, simple manufacturing process, thin and light body, and flexibility. Recently, Samsung and LG have applied OLEDs to mobile phones.

In OLEDs, pros and cons of a material of a luminescent layer decides if it can be used in an industry. Typically, the material of the luminescent layer includes a host luminescent material and a guest luminescent material, and luminescent efficiency and lifetime are crucial indicators for the luminescent material. A ratio of singlet excitons to triplet excitons in OLEDs is 1:3, and conventional fluorescent materials of OLEDs are only able to use the singlet excitons to generate light. Thus, theoretically, internal quantum efficiency of the conventional fluorescent materials is 25%. Phosphorescent metal complex materials, which have been used in red-light OLED displays and green-light OLED displays, are able to use 100% of singlet excitons and triplet excitons due to spin-orbit coupling in heavy metal. However, the phosphorescent metal complex materials usually include costly metals such as Ir, Pt, or Os, which can also be toxic. In addition, the phosphorescent metal complex materials still have issues with efficiency and lifespan.

In 2012, Adachi reported a pure organic luminescent molecular based on a thermally activated delayed fluorescence (TADF) phenomenon. By designing reasonable D-A molecular structures, molecules can have a relatively small difference (ΔEST) between the lowest singlet excited energy level and the lowest triplet excited energy level, which allows the triplet excitons to return to the singlet excitons by a reverse intersystem crossing (RISC). Then, the singlet excitons achieve radiative decay and return to ground state, thereby emitting photons. By using TADF materials, not only can 100% usage of singlet excitons and triplet excitons be achieved, but also heavy metals can be omitted. Moreover, structures of the TADF materials can be diverse, and physical properties thereof are easy to adjust. As a result, organic luminescent materials with high efficiency and long lifespan are realized.

To manufacture OLEDs with high efficiency, the TADF materials applied thereto must have a small ΔEST and a high photoluminescence quantum yield (PLOY). Nowadays, green and blue TADF materials can achieve over 30% external quantum efficiency (EQE). However, for devices using TADF materials with long wavelengths, exceptional performance cannot be realized because of an energy gap law.

An objective of the present disclosure is to solve a following technical problem: in conventional technologies, TADF materials with long wavelengths have a relatively large ΔEST, and devices using them have poor performance.

### SUMMARY

To achieve the above goal, the present disclosure provides a TADF material, including a target compound synthesized from an electron donator and an electron acceptor, wherein the target compound has a Dₙ-A molecular structure, n denotes 1, 2, or 3, D is the electron donator, and A is the electron acceptor.

Furthermore, the target compound has a structure as follows:

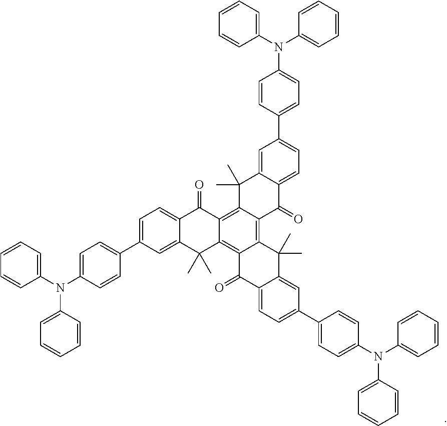 [SMILES: CC1(C)c2cc(-c3ccc(N(c4ccccc4)c4ccccc4)cc3)ccc2C(=O)c2c1c1c(c3c2C(C)(C)c2cc(-c4ccc(N(c5ccccc5)c5ccccc5)cc4)ccc2C3=O)C(C)(C)c2cc(-c3ccc(N(c4ccccc4)c4ccccc4)cc3)ccc2C1=O]

Furthermore, The TADF material of claim 3, wherein the electron acceptor is one of following structures:

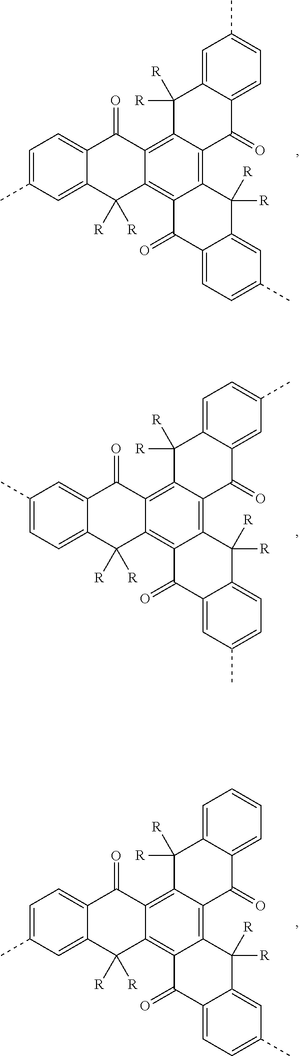

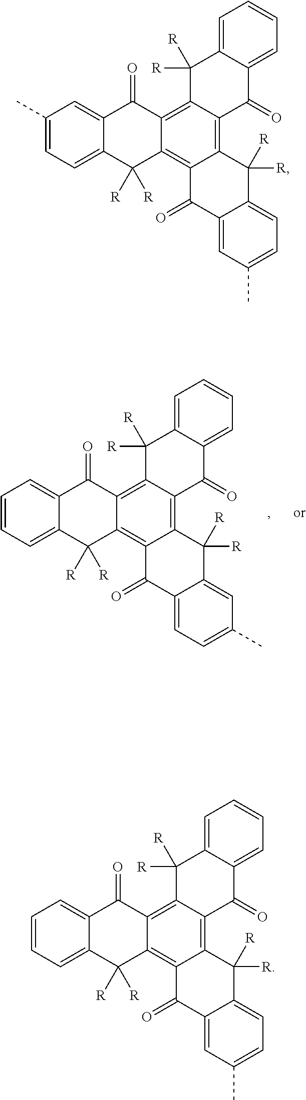

Furthermore, a group R of the electron acceptor is at least one of an alkyl group, an alkoxy group, an aryl group, or a substituted aryl group.

Furthermore, the electron donator is one of following structures or a derivative thereof:

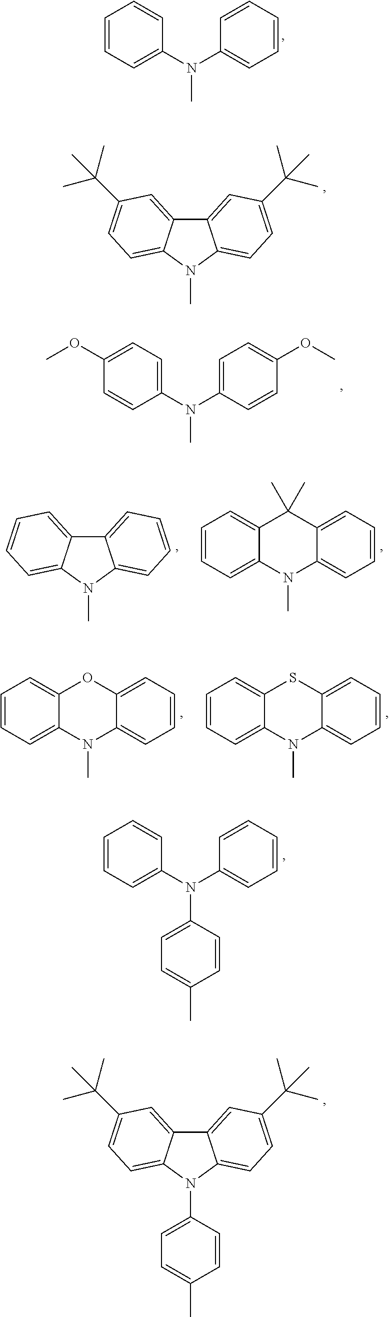

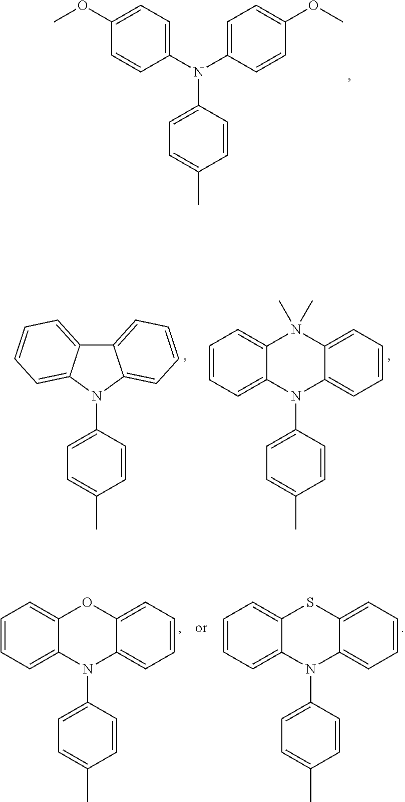

To achieve the above goal, the present disclosure further provides a method of synthesizing a TADF material, including following steps: a reaction solution preparing step: putting an electron donator, an electron acceptor, and a catalyst into a reaction vessel to obtain a reaction solution; a target compound synthesis step: allowing the reaction solution to be fully reacted at a temperature ranging from 50° C. to 100° C. to obtain a mixing solution, wherein the mixing solution includes a target compound formed during the target compound synthesis step; an extraction step: cooling the mixing solution to room temperature, and extracting the target compound from the mixing solution; and a target compound purification step: separating and purifying the mixing compound to obtain the TADF material.

Furthermore, the reaction solution preparing step includes a following step: an electron acceptor solution manufacturing step: putting 2,8,14-tribromo-6,6,12,12,18,18-hexamethyl-naphthyrone and 4-(diphenylamino)-phenylboronic acid into a Schlenk flask, adding toluene and potassium carbonate solution into the Schienk flask, and allowing them to be fully reacted by stirring at room temperature to obtain an electron acceptor solution.

Furthermore, the target compound synthesis step includes a following step: adding tetrakis(triphenylphosphine)palladium(0) into the mixing solution, and allowing them to be fully reacted for 24 hours.

Furthermore, the extraction step includes following steps: cooling the reaction solution to room temperature, and extracting and washing the reaction solution for several times; and combing organic phases after extracting the reaction solution for several times to obtain the target compound. The target compound purification step includes a following step: purifying the target compound by column chromatography with eluent, wherein the eluent includes a 1:1 volume ratio of methylene chloride to petroleum ether.

To solve the above goal, the present disclosure further provides an electroluminescent device, including a substrate layer, and a hole injection layer, a hole transport layer, a luminescent layer, an electron transport layer, and a cathode layer, which are sequentially disposed on the substrate layer, wherein the TADF material described above is used in the luminescent layer.

Regarding the beneficial effects: by combining different functional groups, a series of deep red TADF materials with significant TADF property and relatively high yield are synthesized. The TADF materials account for a high proportion of a synthesized product and has a high PLOY, thereby improving organic electroluminescent devices. The organic electroluminescent devices using the deep red TADF materials have relatively high luminescent efficiency and relatively high brightness.

### DESCRIPTION OF DRAWINGS

Technical solutions and beneficial effects of the present disclosure are illustrated below in detail in conjunction with drawings and a specific embodiment.

FIG. 1 is a flowchart showing a method of synthesizing a TADF material according to an embodiment of the present disclosure.

FIG. 2 is an emission spectrum of the TADF material according to the embodiment of the present disclosure.

FIG. 3 is a schematic structural view showing an electroluminescent device according to the embodiment of the present disclosure.

### DETAILED DESCRIPTION

Hereinafter a preferred embodiment of the present disclosure will be described with reference to the accompanying drawings to exemplify the embodiment of the present disclosure can be implemented, which can fully describe the technical contents of the present disclosure to make the technical content of the present disclosure clearer and easy to understand. However, the described embodiment is only an example of the present disclosure, but not all of the embodiments. All other embodiments obtained by those skilled in the art based on the embodiment of the present disclosure without creative efforts are within the scope of the present disclosure.

In the description of the present disclosure, it should be understood that terms such as “upper”, “lower”, as well as derivative thereof should be construed to refer to the orientation as then described or as shown in the drawings under discussion. These relative terms are for convenience of description, do not require that the present disclosure be constructed or operated in a particular orientation, and shall not be construed as causing limitations to the present disclosure.

In the description of the present disclosure, unless specified or limited otherwise, it should be noted that a structure in which a first feature is “on” or “beneath” a second feature may include an embodiment in which the first feature directly contacts the second feature and may also include an embodiment in which an additional feature is formed between the first feature and the second feature so that the first feature does not directly contact the second feature.

Furthermore, a first feature “on,” “above,” or “on top of” a second feature may include an embodiment in which the first feature is right “on,” “above,” or “on top of” the second feature and may also include an embodiment in which the first feature is not right “on,” “above,” or “on top of” the second feature, or just means that the first feature has a sea level elevation greater than the sea level elevation of the second feature.

Furthermore, a first feature “beneath,” “below,” or “on bottom of” a second feature may include an embodiment in which the first feature is right “beneath,” “below,” or “on bottom of” the second feature and may also include an embodiment in which the first feature is not right “beneath,” “below,” or “on bottom of” the second feature, or just means that the first feature has a sea level elevation less than the sea level elevation of the second feature.

The disclosure below provides many different embodiments or examples for realizing different structures of the present disclosure. In order to simplify the disclosure of the present disclosure, components and settings of specific examples are described below. Of course, they are only examples and are not intended to limit the present disclosure. Furthermore, reference numbers and/or letters may be repeated in different examples of the present disclosure. Such repetitions are for simplification and clearness, which per se do not indicate the relations of the discussed embodiments and/or settings.

An embodiment of the present disclosure provides a TADF material which is a target compound synthesized from an electron donator and an electron acceptor. The target compound has a Dₙ-A molecular structure, wherein n denotes 1, 2, or 3, D is the electron donator, and A is the electron acceptor.

The target compound has a structure as follows:

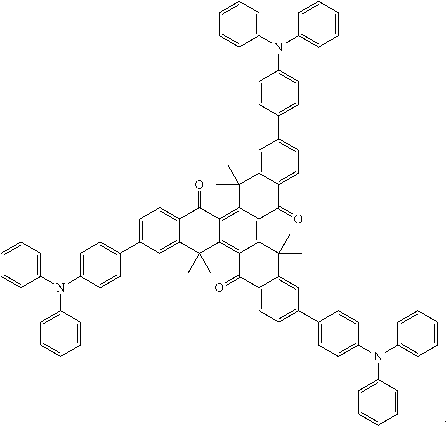 [SMILES: CC1(C)c2cc(-c3ccc(N(c4ccccc4)c4ccccc4)cc3)ccc2C(=O)c2c1c1c(c3c2C(C)(C)c2cc(-c4ccc(N(c5ccccc5)c5ccccc5)cc4)ccc2C3=O)C(C)(C)c2cc(-c3ccc(N(c4ccccc4)c4ccccc4)cc3)ccc2C1=O]

The electron acceptor is one of following structures:

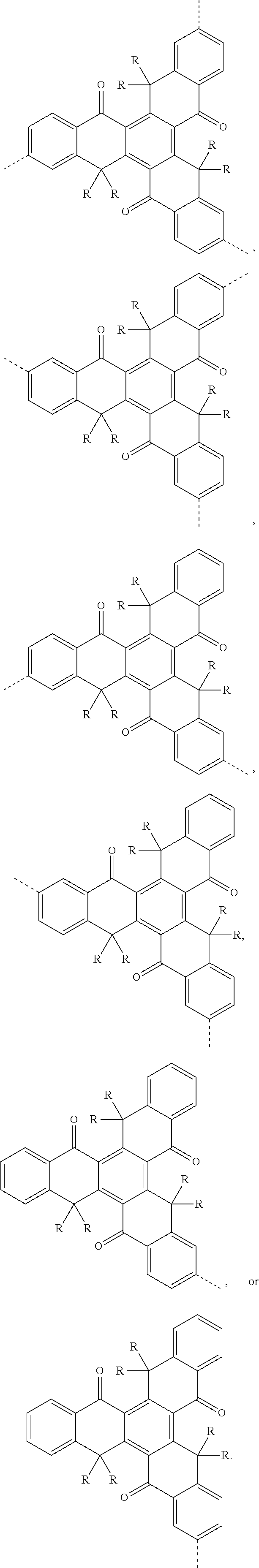

A group R of the electron acceptor is at least one of an alkyl group, an alkoxy group, an aryl group, or a substituted aryl group.

The electron donator is one of following structures or a derivative thereof:

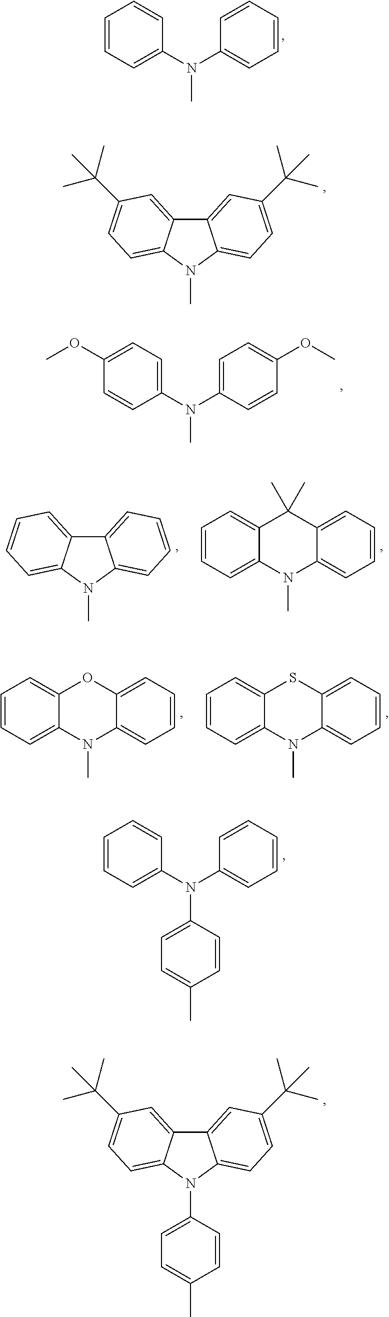

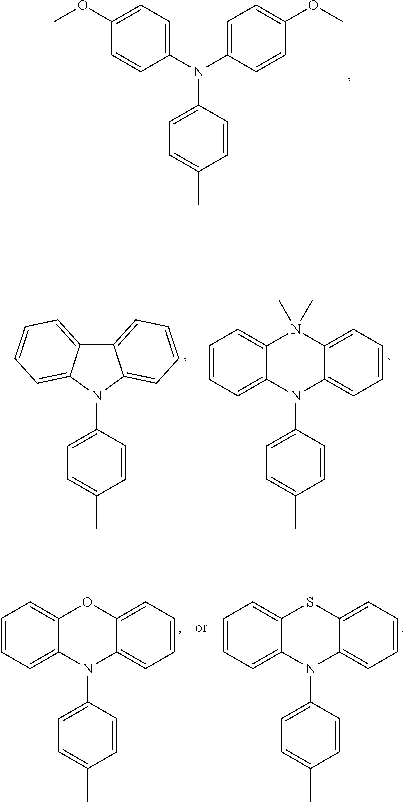

TABLE 1
parameters, such as the lowest singlet state (S1) and
the lowest triplet state (T1), of the target compound

| | PL Peak | | | ΔEST | PLQY |
|---|---|---|---|---|---|
| | (nm) | S1 (eV) | T1 (eV) | (eV) | (%) |
| Target compound | 636 | 2.25 | 2.16 | 0.09 | 94 |

FIG. 2 shows a photoluminescence spectrum of the TADF material in a toluene solution at room temperature.

Typically, long-wavelength TADF materials have a molecular structure consisting of an electron donator (D) and an electron acceptor (A). The electron acceptor (A) has a large plane and is rigid, which can reduce a non-radiative transition of molecules, thereby increasing a rate of radiative transition of the molecules and achieving high PLOY. In the present embodiment, the long-wavelength TADF materials including trimeric naphthalenone have the electron acceptor (A) including a carbonyl structure and a very large plane, which allows molecules to have a high intersystem crossing rate constant and a high anti-intersystem crossing rate constant. Therefore, a reduction of radiative transition rate due to an energy gap law can be restrained, thereby achieving high PLOY. Furthermore, the molecules have good rigidity due to the large plane, which increases stability of the TADF materials and extends lifetime of devices.

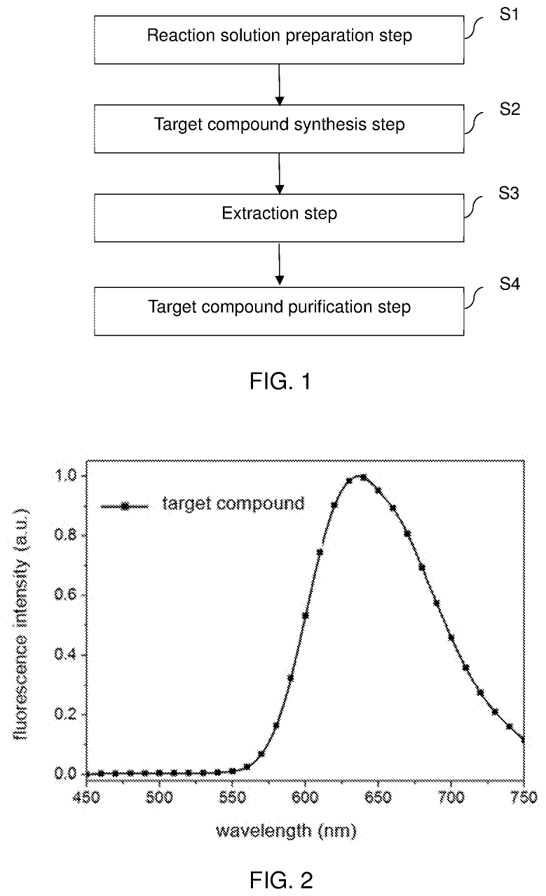

As shown in FIG. 1, the present embodiment further provides a method of synthesizing a TADF material, including steps S1 to S4, and a synthesis route is shown below:

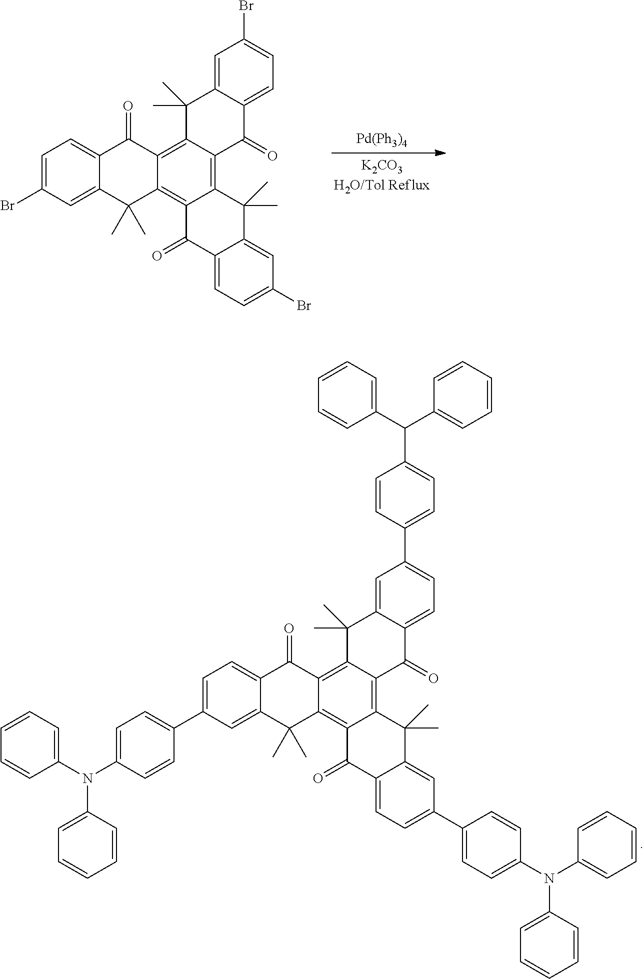 [SMILES: CC1(C)c2cc(Br)ccc2C(=O)c2c1c1c(c3c2C(C)(C)c2cc(Br)ccc2C3=O)C(C)(C)c2cc(Br)ccc2C1=O ; CC1(C)c2cc(-c3ccc(C(c4ccccc4)c4ccccc4)cc3)ccc2C(=O)c2c1c1c(c3c2C(C)(C)c2cc(-c4ccc(N(c5ccccc5)c5ccccc5)cc4)ccc2C3=O)C(C)(C)c2cc(-c3ccc(N(c4ccccc4)c4ccccc4)cc3)ccc2C1=O ; "Pd(Ph3)4"]

S1: a reaction solution preparation step: putting an electron donator, an electron acceptor, and a catalyst into a reaction vessel to obtain the reaction solution. Specifically, putting 8 mmol to 12 mmol of 2,8,14-tribromo-6,6,12,12,18,18-hexamethyl-trinaphthalenone and 30 mmol to 35 mmol of 4-(diphenylamino)-phenylboronic acid into a 100 ml to 200 ml Schlenk flask, adding 25 ml to 35 ml of toluene and 8 ml to 12 ml of potassium carbonate solution into the Schlenk flask, allowing them to be fully reacted by stirring at room temperature, and substituting gas in the Schlenk flask with argon to obtain an electron acceptor solution. In the present embodiment, after putting tribromo-6,6,12,12,18,18-hexamethyl-trinaphthalenone (7.47 g, 10 mmol) and 4-(diphenylamino)-phenylboronic acid (3.18 g, 33 mol) into a Schlenk flask, toluene (30 ml) and potassium carbonate solution (2.5M, 10 ml) are added into the Schlenk flask. Then, a solution in the Schlenk flask is fully reacted by stirring at room temperature, and gas in the Schlenk flask is substituted by argon. Finally, the electron acceptor solution is obtained.

S2: a target compound synthesis step: allowing the reaction solution to be fully reacted at a temperature ranging from 50° C. to 100° C. to obtain a mixing solution, wherein the mixing solution includes the target compound formed during the target compound synthesis step, and then adding 0.2 mmol to 0.5 mmol of tetrakis(triphenylphosphine)palladium(0) into the mixing solution, and allowing them to be fully reacted for 24 hours. In the present embodiment, the reaction solution is fully reacted at 80° C. to obtain the mixing solution including the target compound formed during the target compound synthesis step. After adding 0.4 mmol of tetrakis(triphenylphosphine)palladium(0) into the mixing solution, a reflux reaction is fully performed at 80° C. for 24 hours.

S3: an extraction step: cooling the mixing solution to room temperature, and extracting the target compound from the mixing solution. Specifically, cooling the mixing solution to room temperature, and extracting the target compound from the mixing solution with methylene chloride several times. In the present embodiment, the target compound is extracted from the mixing solution and washed with water three times preferably. After that, organic phases are combined to obtain the target compound.

S4: a target compound purification step: separating and purifying the mixing compound to obtain the TADF material. Specifically, purifying the target compound by silica gel column chromatography with eluent, wherein the eluent includes a 1:1 volume ratio of methylene chloride to petroleum ether. The methylene chloride is a solvent in the column chromatography. After the target compound is separated and purified, a deep red powder (11.40 g) can be obtained, and a yield rate is 92%. Results of H-NMR and C-NMR are shown as follows: HRMS [M+H]+ calcd. for C₉₀H₆₉N₃O₃: 1239.5339; found: 1239.5356.

Regarding the beneficial effects: by combining different functional groups, a series of deep red TADF materials with significant TADF property and relatively high yield are synthesized. The TADF materials account for a high proportion of a synthesized product, and has a high PLOY.

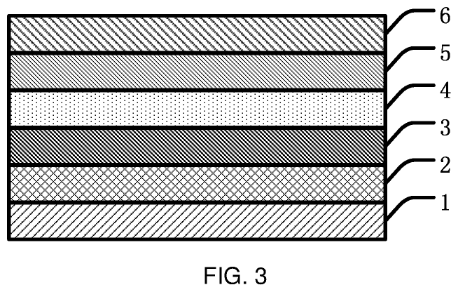

As shown in FIG. 3, the present embodiment further provides an electroluminescent device, including a substrate layer 1, and a hole injection layer 2, a hole transport layer 3, a luminescent layer 4, an electron transport layer 5, and a cathode layer 6, which are sequentially disposed on the substrate layer 1, wherein the TADF material mentioned above is applied to the luminescent layer 4.

Specifically, a layer of 2,3,6,7,10,11-hexacyano-1,4,5,8,9,12-hexaazatriphenylene (HATCN) (30 nm) is spin-coated on a water-washed substrate layer 1 to form the hole injection layer 2, wherein a material of the substrate layer 1 is indium tin oxide (ITO).

A layer of 4,4′-cyclohexylidenebis[N,N-bis(4-methylphenyl)benzenamine] (TAPC) (40 nm) is spin-coated on the hole injection layer 2 to form the hole transport layer 3.

A layer of the TADF material (40 nm) mentioned above and 5% host material, 4,4′-bis(N-carbazolyl)-1,1′-biphenyl (CBP), are spin-coated to form the luminescent layer 4.

Under high vacuum conditions, a layer of 1,3,5-tris(3-pyridyl-3-phenyl)benzene (Tm3PyPB) (40 nm) is vapor-deposited on the luminescent layer 4 to form the electron transport layer 5.

Under high vacuum conditions, a layer of LiF (1 nm) and Al (100 nm) is vapor-deposited on the electron transport layer 5 to form the cathode layer 6. Finally, the electroluminescent device is formed.

A current-brightness-voltage characteristic of the electroluminescent device was measured by Keithley's source measurement systems (Keithley 2400 Sourcemeter and Keithley 2000 Currentmeter) with calibrated silicon photodiode. An Electroluminescence spectroscopy was measured by a spectrometer (SPEX CCD 3000) of the French JY company. All measurements were performed at room temperature under atmosphere.

The characteristics of the electroluminescent device are shown in Table. 2 as follows.

TABLE 2
parameters, such as the highest brightness and the
highest EQE, of the electroluminescent device

| | Highest | | |
|---|---|---|---|
| | brightness | EL peak | Highest EQE |
| Device | (cd/m2) | (nm) | (%) |
| Electroluminescent | 3395 | 642 | 24 |
| device | | | |

The electroluminescent device adopting the deep red TADF material has relatively high luminescent efficiency, high brightness, high manufacturing efficiency, and long lifetime.

Regarding the beneficial effects: by combining different functional groups, a series of deep red TADF materials with significant TADF property and relatively high yield are synthesized. The TADF materials account for a high proportion of a synthesized product, and has a high PLOY, thereby improving organic electroluminescent devices. The organic electroluminescent devices using the deep red TADF materials have relatively high luminescent efficiency and relatively high brightness.

In the above embodiments, the focus of each embodiment is different, and for a part that is not detailed in an embodiment, reference may be made to related descriptions of other embodiments.

A TADF material, a synthesizing method thereof, and an electroluminescent device have been described in detail with embodiments provided by the present disclosure which illustrate principles and implementations thereof. However, the description of the above embodiments is only for helping to understand the technical solution of the present disclosure and core ideas thereof, and it is understood by those skilled in the art that many changes and modifications to the described embodiments can be carried out without departing from the scope and the spirit of the disclosure that is intended to be limited only by the appended claims.

What is claimed is:

1. A thermally activated delayed fluorescence (TADF) material, comprising a target compound synthesized from an electron donator and an electron acceptor, wherein the target compound has a Dₙ-A molecular structure, n denotes 1, 2, or 3, D is the electron donator, and A is the electron acceptor.

2. The TADF material of claim 1, wherein the target compound has a structure as follows:

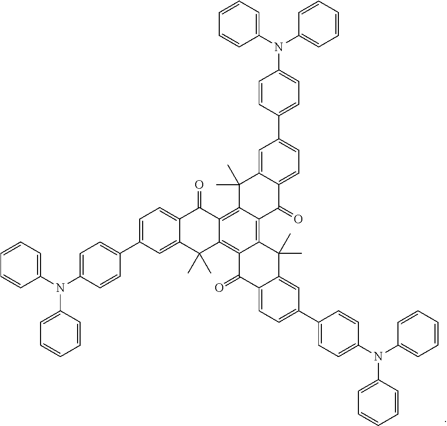 [SMILES: CC1(C)c2cc(-c3ccc(N(c4ccccc4)c4ccccc4)cc3)ccc2C(=O)c2c1c1c(c3c2C(C)(C)c2cc(-c4ccc(N(c5ccccc5)c5ccccc5)cc4)ccc2C3=O)C(C)(C)c2cc(-c3ccc(N(c4ccccc4)c4ccccc4)cc3)ccc2C1=O]

3. The TADF material of claim 1, wherein the electron acceptor is one of following structures:

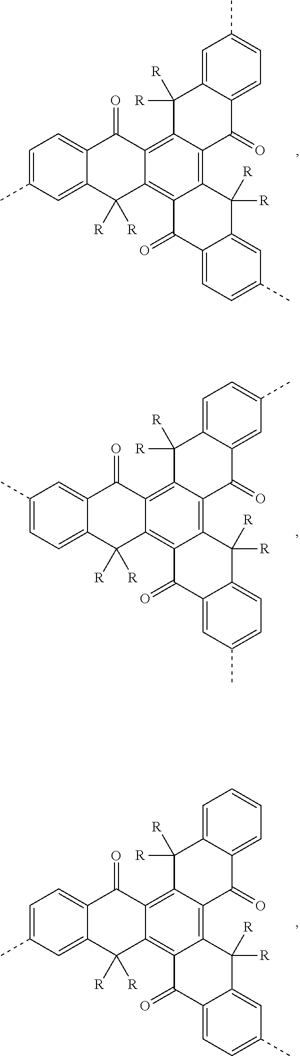

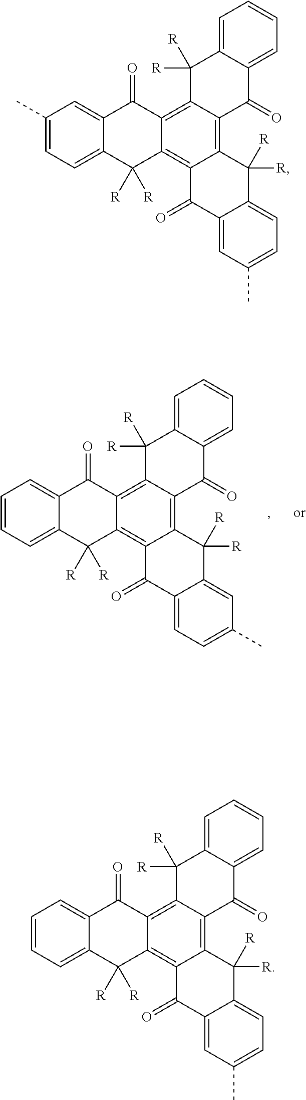

4. The TADF material of claim 3, wherein a group R of the electron acceptor is at least one of an alkyl group, an alkoxy group, an aryl group, or a substituted aryl group.

5. The TADF material of claim 1, wherein the electron donator is one of following structures or a derivative thereof:

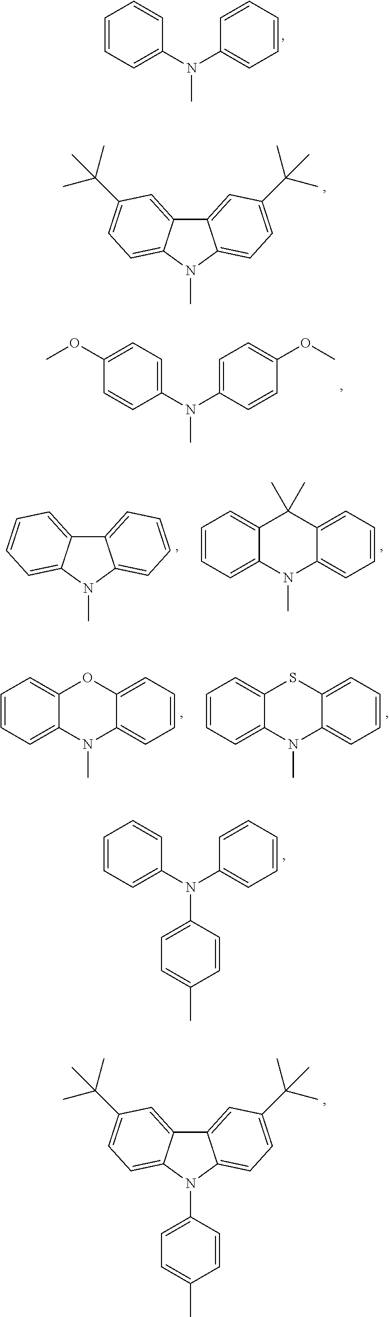

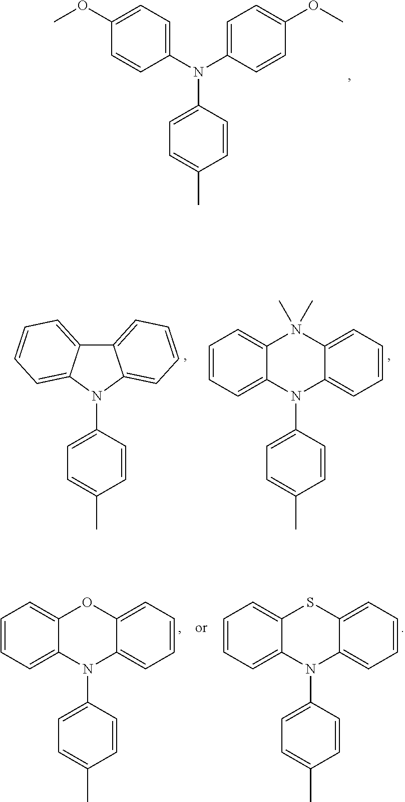

6. A method of synthesizing a thermally activated delayed fluorescence (TADF) material, comprising following steps:
a reaction solution preparing step: putting an electron donator, an electron acceptor, and a catalyst into a reaction vessel to obtain a reaction solution;
a target compound synthesis step: allowing the reaction solution to be fully reacted at a temperature ranging from 50° C. to 100° C. to obtain a mixing solution, wherein the mixing solution comprises a target compound formed during the target compound synthesis step;
an extraction step: cooling the mixing solution to room temperature, and extracting the target compound from the mixing solution; and
a target compound purification step: separating and purifying the mixing compound to obtain the TADF material.

7. The method of claim 6, wherein the reaction solution preparing step comprises a following step:
an electron acceptor solution manufacturing step: putting 2,8,14-tribromo-6,6,12,12,18,18-hexamethyl-trinaphthalenone and 4-(diphenylamino)-phenylboronic acid into a Schlenk flask, adding toluene and potassium carbonate solution into the Schlenk flask, and allowing them to be fully reacted by stirring at room temperature to obtain an electron acceptor solution.

8. The method of claim 6, wherein the target compound synthesis step comprises a following step:
adding tetrakis(triphenylphosphine)palladium(0) into the mixing solution, and allowing them to be fully reacted for 24 hours.

9. The method of claim 6, wherein the extraction step comprises following steps:
cooling the reaction solution to room temperature, and extracting and washing the reaction solution with water several times; and
combing organic phases after extracting the reaction solution for several times to obtain the target compound; and
wherein the target compound purification step comprises a following step:
purifying the target compound by silica gel column chromatography with eluent, wherein the eluent comprises a 1:1 volume ratio of methylene chloride to petroleum ether.

10. An electroluminescent device, comprising a substrate layer, and a hole injection layer, a hole transport layer, a luminescent layer, an electron transport layer, and a cathode layer, which are sequentially disposed on the substrate layer, wherein the TADF material of claim 1 is used in the luminescent layer.
